(19) United States
(12) Patent Application Publication
(10) Pub. No.: US 2026/0023063 A1
(43) Pub. Date: Jan. 22, 2026

(54) Photoacoustic method for characterizing a plant

(71) Applicant: COMMISSARIAT À L'ENERGIE ATOMIQUE ET AUX ENERGIES ALTERNATIVES, PARIS (FR)

(72) Inventors: Kévin JOURDE, GRENOBLE (FR); Héloïse HANNART, GRENOBLE (FR)

(21) Appl. No.: 19/265,962

(22) Filed: Jul. 10, 2025

(30) Foreign Application Priority Data
Jul. 19, 2024 (FR) 2407966

(51) Int. Cl. G01N 33/00; G01N 21/17; G01N 21/84

(52) U.S. Cl. CPC G01N 33/0098; G01N 21/1702; G01N 21/84; G01N 2021/8466

(57) ABSTRACT

A photoacoustic method for characterizing a plant, the method includes applying laser radiation to a material surface of the plant, recording sound waves appearing under the photothermal effect in a photoacoustic cavity positioned around the surface of the plant using a microphone, with the laser radiation having been applied to the surface through the cavity. The plant surface is a surface of a stem (or a root or a petiole) of the plant, and the method includes inserting a section of the stem into a support including the cavity equipped with a microphone sensor and a window for transmitting laser power, with sealing means being installed around the stem at each of the two ends of the A section.

### CROSS-REFERENCE TO RELATED APPLICATION

This application claims priority to foreign French patent application No. FR 2407966, filed on Jul. 19, 2024, the disclosure of which is incorporated by reference in its entirety.

### FIELD OF THE INVENTION

The invention is in the field of devices and methods for exploring living organisms, notably plants, and more specifically for monitoring plant activity in vivo.

### BACKGROUND

It uses photoacoustics, also called optoacoustics, i.e., the observation of mechanical waves induced in a body illuminated by electromagnetic radiation. The phenomena underlying this generation relate to photothermy, whereby incident waves heat the body, and thermal expansion, whereby an increase in temperature causes expansion. Incident light that varies over time is used to create expansion, which is followed by contraction.

The ability to absorb light depends on the constitution of the material and the electromagnetic wave. The use of photoacoustics is therefore suitable for spectroscopy manipulations. Of particular interest is a spectroscopy approach in the mid-infrared (MIR) spectral range. This range of the electromagnetic spectrum offers a specific signature of molecules. The acquisition of spectra then allows the molecular composition of the studied bodies to be traced and optionally allows the chemical species that are present to be quantified.

In recent years, work has primarily focused on the development of miniature gas sensors based on this principle of mid-infrared spectroscopy by photoacoustic transduction. These developments were then applied to the characterization of liquids and solids and this led to the development, for example, of a sensor capable of non-invasively monitoring the blood sugar level of an individual.

Mid-infrared spectrometry without acoustic transduction is also used in the field of plant biology. Indeed, work already carried out on plants uses Fourier transform infrared (FTIR) microspectrometry, which allows maps to be established on a scale of tens of microns of plant tissues sampled in strips or reduced to powder, for example for analyzing the composition of cell walls, which is of interest for understanding intracellular communications and, in practical terms, for the wood industry.

Research has also been conducted to develop the use of photoacoustics for studying plants, again using infrared spectroscopy. It has been proposed for the modulation frequency of the optical beam to be modified in order to deduce the chemical composition within the thickness of plant leaf samples.

The use of open cavities affixed to plant leaves has been proposed. Such arrangements for in vivo studies of photosynthesis are also known from Pereira et al., Meas Sci. Technol. 3, 1992, 931, as well as from Mesquita et al., Instrumentation science and technology, Vol. 34, 33, 2006, which involve positioning an open cavity with its mouth pressed against a plant leaf, thereby closing the cavity. The other side of the leaf is exposed to visible light (white light and monochromatic light at 680 nm or 650 nm), guided by optical fiber. The cavity contains an electret microphone. These experiments, carried out with visible wavelengths, aim to monitor photosynthetic activity in vivo. However, the measurement is invasive (the leaf is covered) and therefore does not allow prolonged monitoring without affecting plant activity.

In the article by Helfter C. et al., (2007) Tree Physiology, Vol. 27, 169, the response of an intact plant stem to a near-infrared (812 nm) heat source is observed using an infrared camera. The approach is non-invasive, but the characterization does not provide access to chemical information.

It has also been proposed for living tissues to be examined in the mid-infrared range using attenuated total reflectance FTIR (ATR-FTIR) in Zhang et al., 2023, Scientific Reports, Vol. 13. The limitations of the method set forth in this article relate to the temporal monitoring, which cannot be precise without disturbing the plant, and to the examined area being restricted to the first few microns of the sample.

Finally, in Gaoqiang L., et al., 2020, Spectrochimica Acta Part A, Vol. 228, an approach using mid-infrared photoacoustic spectrometry to find the chemical composition in plant leaf samples has been set forth.

### SUMMARY OF THE INVENTION

The remainder of the description will now propose applying a photoacoustic cavity on the stem of a plant for the study and monitoring thereof.

In order to overcome the shortcomings of these prior art systems and methods, a photoacoustic method for characterizing a plant is proposed, with the method comprising applying laser radiation to a material surface of the plant, recording sound waves appearing under the photothermal effect in a photoacoustic cavity positioned around said surface of the plant using a microphone, with the laser radiation having been applied to said surface through the cavity.

Furthermore, remarkably, the plant surface is a surface of a stem, a root or a petiole of the plant, with the plant being intact and typically alive and growing, and the method comprises inserting a section of said stem, root or petiole into a support comprising the cavity equipped with a microphone sensor and a window for transmitting laser power from the laser beam, with sealing means being installed around the stem, root or petiole at each of the two ends of said section. This method is non-invasive.

These principles allow the stem, the root or the petiole of a plant, for example a stem with a diameter of 2 to 3 mm, to be explored, and notably allow the phloem and the xylem to be probed at various wavelengths throughout the life of the plant.

Various optical sources for photoacoustics can be used. Monitoring over a time scale relevant to the changes in the plant allow the associated dynamics to be known and appropriate actions to be taken.

More specifically, a system is proposed that includes a sensor for monitoring plant activity using mid-infrared photothermal monitoring.

By virtue of mid-infrared (MIR) photothermal monitoring, the full measure of spectroscopy can be applied to the mid-infrared range, with photothermal monitoring allowing the thickness, typically around a hundred microns, of the sample to be scanned, with the exact achievable depth depending on the chemical composition of the studied plant and the range of wavelengths used in the MIR. This then allows temporal monitoring resolutions of around one second to be obtained over long time scales, for example several days.

A mid-infrared spectroscopy measurement is therefore performed on the wall of the stem, root or petiole of a plant. This measurement is performed in vivo, is minimally invasive, and can be quasi-continuous. It allows a signal specific to plant activity to be detected with a very high probability. This measurement has significant potential for helping to understand the processes taking place in plants, which opens up a wide field of fundamental exploration and practical applications.

Preferably, the environment is measured simultaneously and the optical power of the laser is measured continuously. In both cases, this is carried out in order to correct the measurements or to identify correlations, which may or may not be specific to the activity of the studied plant.

The multi-frequency modality of photoacoustics allows the characterization depth of the sample to be varied. Thus, it allows the depth of the sample to be scanned and optionally allows a distinction to be made between processes occurring at different depths but within the outermost layer of 150 μm of the stem, the root or the petiole. Thus, advantageously and optionally:
the support can be made up of a set of two opposing jaws forming a vice and each provided with a seal;
the laser radiation can be applied as a continuous wave with amplitude modulation at several frequencies; modulation frequencies thus can be selected, for example from 100 to 1,000 Hz, which allows some of them to probe deeper layers of the stem, root or petiole, and others to probe more superficial layers;
the laser radiation can be at a frequency in the mid-infrared range corresponding to an absorption band of a molecule of interest for monitoring the growth of the plant. For example, glucose at 1,036 cm⁻¹ may be of interest. The laser radiation also can be made up of a set of laser sources at different optical wavelengths relevant for plant analysis;
the recording can be carried out for a period of around 1 second, with sampling at a frequency of around 100 kHz, but in general a photoacoustic spectrum (with several modulation frequencies) is recorded with one or more lasers, with the way in which this spectrum is recorded being adapted to the studied case. The spectrum can be recorded by taking several measurements at different modulation frequencies (with the duration then depending on the desired signal-to-noise ratio), or in pulse mode;
the method can involve continuously monitoring the plant by regularly repeating the application of the laser and the associated subsequent measurement for at least one day and one night;
the measurement can be demodulated in order to identify a phase and a modulus, and thus generate a photoacoustic spectrum;
the plant can be alive and is hardly affected by the measurement; this is a major advantage of the method: the respiration or photosynthesis activity is not disturbed, and the measurement still can be carried out over a very long period of time in a non-invasive manner.

The invention also relates to photoacoustic equipment for characterizing a plant, the equipment comprising means for applying laser radiation, a photoacoustic cavity configured so that the laser radiation can be applied through the cavity, and means for recording sound waves appearing under the photothermal effect in the photoacoustic cavity by means of a microphone.

The equipment further comprises a support comprising the cavity, with the support also comprising an insertion space for a section of a plant stem, root or petiole and sealing means for sealing the cavity at each of the two ends of said section. The seals are preferably specifically adjusted to the dimensions of the tested stem, root or petiole. The means for applying laser radiation can further comprise a tunable laser or a plurality of optically coupled monochromatic lasers.

A system is thus developed that can be permanently installed on the plant without affecting its development.

There are numerous applications, notably related to the climate crisis and the need to monitor plant activity in order to help the agri-food sector understand the state of its production and to anticipate how plants will adapt to the environmental changes they will experience, as well as to the likely future shortage of available resources.

Optionally, the support is made up of a set of two opposing jaws forming a vice and each provided with a thin circular seal in order to form, with the opposing seal, a set of two sealed opposing passages on either side of the cavity for positioning the stem, the root or the petiole, with a seal around the stem, the root or the petiole.

### BRIEF DESCRIPTION OF THE DRAWINGS

FIG. 1 shows a diagram of the device of the invention.

FIGS. 2 to 5 show three-dimensional views of an embodiment of the device according to the invention.

FIG. 6 shows a plant stem and the spaces therein that are explored using the principles of the invention.

FIGS. 7 and 8 show measurements obtained with the device according to the invention.

### DETAILED DESCRIPTION

The intention is to monitor the growth dynamics of a plant, for example the dynamics for conveying and storing sugars, and to thus predict its efficiency, for example so that the agricultural sector can achieve production with higher nutritional quality while minimizing external provisions.

A further intention is to detect the presence of chemicals linked to changes in the air or soil, as plants are a kind of concentrator that can be used to detect subtle variations in the environment. Specifically, the intention is to temporally monitor the relevant chemical compound in the plant over time. To this end, the absorption spectrum associated with this compound is used, as well as the spectra of compounds that are likely to generate an equivalent photoacoustic signal. For the one or more wavelengths that appear most suitable for detecting the compound, the amplitude of the signal associated with variations in its concentration is evaluated, ruling out any signal variations linked to variations in the concentration of any interfering compounds.

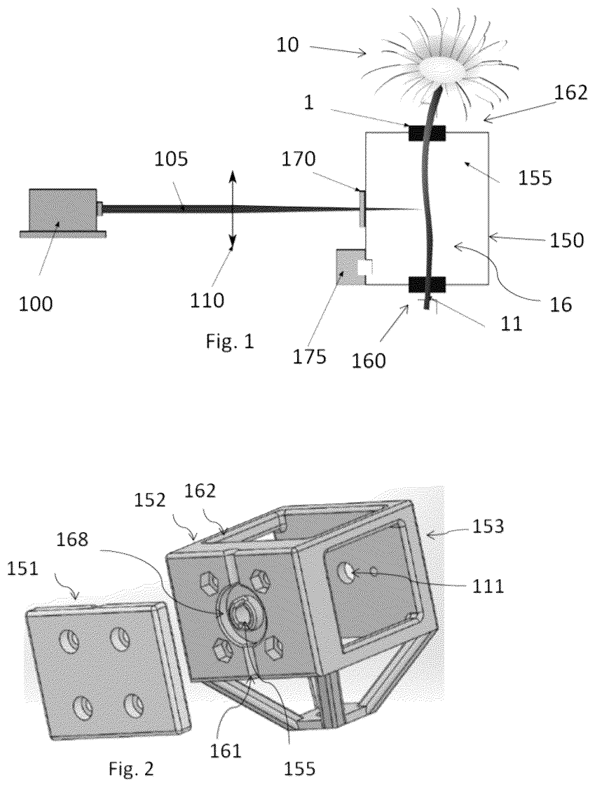

FIG. 1 shows an assembly implementing the principles of the invention for monitoring the plant 10.

A laser 100, which is a quantum cascade laser emitting in the mid-infrared range, produces a laser beam 105 that is processed by a converging lens 110.

The optical source is a quantum cascade laser (QCL) emitting in the mid-infrared range, in a restricted spectral range, or in a wider range with the possibility of wavelength tuning the source. A specific case is that of a monochromatic laser of the QCL DFB (Distributed Feedback Laser) type. Another example is an external cavity QCL laser. It is also possible to have several QCL and/or DFB lasers coupled by an optical coupler system, then switched on intermittently.

A sealed housing 150 defines a cavity 155 with two openings 160 and 162 facing each other on two opposite walls of the sealed housing 150. The sealed housing 150 is formed in two parts (not shown in the figure) that can move relative to each other and join together to form the shell of the cavity and each of the two openings 160 and 162. One of the two parts comprises a window 170 transparent to waves in the mid-infrared range, for example a silicon or germanium window with a suitable anti-reflective treatment. One of the two parts comprises, oriented toward its convexity, a microphone 175 (mounted on an electronic board) for performing the photoacoustic measurements in the cavity and with an electrical power supply from outside the housing and means for sending the captured data to an external computer. The part of the housing that accommodates the microphone 175 and its electronic board can be the part that accommodates the window 170, as shown in the figure, but it also can be the other part.

The plant 10 has a stem 11 separating the lower parts, typically the roots, on the one hand, from the upper parts, typically the leaves, fruit and flowers, on the other hand. The stem is positioned, for monitoring the plant 10, so as to pass through the openings 160 and 162 before the two movable parts are joined in order to form the cavity. Sealing means are also positioned around the stem 11, in each of the openings 160 and 162, so as to provide a seal around said stem. A section 16 of the stem extends between the two openings 160 and 162; said section 16 should be longer than the height separating the openings 160 and 162. The measurement can be taken not only on a stem, but also on any cylindrical part of the plant: stem, petiole or root.

The laser beam 105 is directed and the lens 110 is positioned so that the laser power passes through the window 170 and is focused on the stem 11, in the cavity 155.

The photoacoustic reaction of the plant is measured over a long period of time, and the environment is measured simultaneously, and the optical power of the laser is also continuously measured. The measurements of the reaction of the plant are corrected according to the environment and the laser, and correlations are sought. The provisions given to the plant to improve crop yield are also corrected.

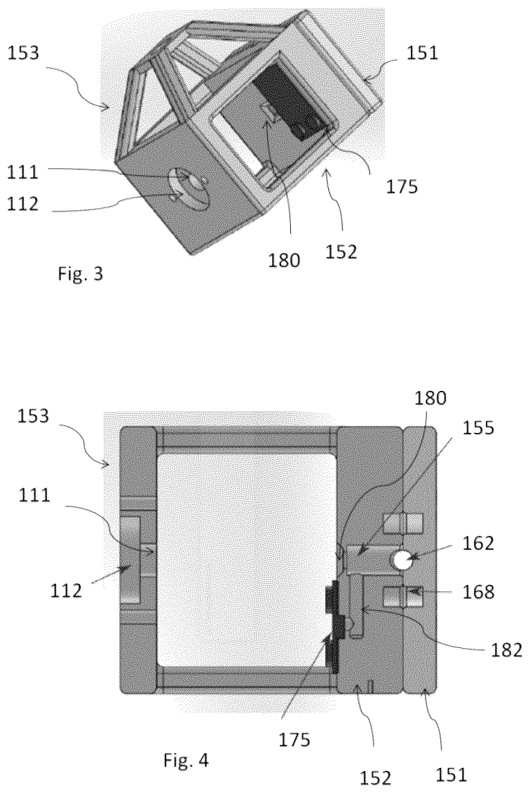

FIG. 2 The following figures show several three-dimensional views of a photothermal device for indirect acoustic detection (the acoustic wave is transmitted by the gas in the cavity, in this case air, to the microphone), in accordance with the principles of the invention.

The photoacoustic cavity is positioned like a vice, i.e. clamped, on the stem 11 of the plant and the stem is thus located in the cavity. The cavity is sealed by suitable means with limited impact on the plant. For example, the sealing means are silicone seals specifically designed for the diameter of the stems to be analyzed. These silicone seals allow the photoacoustic cell to be sealed without damaging the plant. The microphone detects the acoustic wave induced by the photothermal effect in the photoacoustic cavity.

The sealed housing 150 is made up of two jaws 151 and 152, each forming one of the aforementioned housing parts. In FIG. 2, the two jaws are positioned opposite each other without being in contact with each other.

The jaw 151 assumes a general shape of a rectangular plate, one face of which (not shown in FIG. 2) has recesses for forming the cavity 155, on the one hand, and the openings 160 and 162, on the other hand. The other face (shown in FIG. 2 and which can be described as the rear face) is less functional and will not be discussed herein.

The other jaw, namely the jaw 152, also assumes a general shape of a rectangular plate, thicker than the plate of the jaw 151, but with the same lateral dimensions as the rectangle of the jaw 151, with the two jaws 151 and 152 being assembled by placing the sides with the same dimensions opposite each other.

The jaw 152 has recesses on one face (shown in FIG. 2) for forming, with corresponding recesses in the other jaw, the cavity 155 and the openings 160 and 162. On its other face (not shown in FIG. 2, and which can be described as the rear face), the jaw has an offset structure 153 that is large enough, on the one hand, for optically processing light from the laser and, on the other hand, for fixing the housing, for example with an arm or a foot.

The cavity 155 is a small space relative to the entire construction formed by the two jaws. It is formed by recesses in the two plates, in the center of the rectangles forming the faces of these plates, which are joined together once the cavity has been formed. The recesses in the two jaws are different sizes: the cavity is more developed on one side of the abutment plane of the two jaws than on the other, in this case in the jaw 152.

More specifically, the cavity 155 is a space delimited by a wall in the shape of a rotary cylinder with an axis perpendicular to the planes of the two plates. This wall is formed in the jaw 152. The cavity is also delimited by bases, one in one jaw and the other opposite one in the other jaw.

The openings 160 and 162 are defined, for their part, by corresponding recesses in the two plates, which together assume the shape of a rotary cylinder with an axis parallel to the plane of the plates and, more specifically, in the embodiment shown in the figure, parallel to the short side of the rectangles forming the jaws. These recesses forming the openings 160 and 162 are formed half in one jaw and half in the other jaw. The two openings 160 and 162 have the same axis and the same diameter, with the cavity 155 being located halfway between the first opening and the second opening. The openings emerge on both sides of the interface between the two jaws.

The face of the jaw 152 also includes an annular recess around the cylindrical wall forming the cavity 155. Its counterpart is located in the face of the jaw 151 against which the jaw 152 is applied when the cavity is closed, so as to form an annular space 168 for installing an O-ring between the two jaws. The annular space 168 meets the openings 161 and 162 and, in the extension thereof, the cylindrical wall separating the cavity 155 from the annular space 168 is perforated by a cut-out that extends the openings 161 and 162 so that the stem of the plant can be placed in the openings 161 and 162, while extending into the cavity and passing through the annular space 168 twice, on either side of the cavity 155.

The offset structure 153 also includes a through opening 111 for the passage of laser light that is located in the extension of the cavity 155.

In FIG. 3, which like FIG. 2 is a three-quarter view, the two jaws are positioned opposite each other and in contact with each other, thus forming the cavity, which is therefore invisible. The photoacoustic sensor or microphone 175 and its electronic board for monitoring plant activity are shown. This equipment is embedded in the jaw 152 and is flush with the rear face thereof. A window 180 made of a material transparent to the wavelengths of the laser is also shown, flush with the center of the rear face of the jaw 152, not far from the electronic board of the microphone 175.

The offset structure 153 has a mounting space 112 for the converging slot that is visible from the viewing angle. The through opening 111 for the passage of laser light is located behind this mounting space 112. The window 180, the mounting space 112 and the opening 111 are aligned.

In FIG. 4, the configuration of FIG. 3 is shown as a side view, in the direction of the openings 161 and 162, which are aligned perpendicular to the sectional view in a plane passing through the cavity 155 in a plane coinciding with the optical axis.

The microphone 175 is visible with its electronic board, as are the through opening 111 for the passage of the laser light and the mounting space 112 for the converging lens.

The annular space 168 appears in two fragments, on either side of the cavity 155. It is symmetrical on either side of the interface between the two jaws (like the openings 161 and 162, but unlike the cavity 155, which extends further into the jaw 152).

The cavity 155 is visible: it extends over approximately four-fifths of the thickness of the jaw 152, between the abutment surface of the two jaws and the window 180 made of a material transparent to the laser wavelengths, which window is present in the center of the rear face of the jaw 152 in the extension of the cylinder forming the cavity 155.

The jaw 152 further comprises a conduit 182 in its internal volume that allows sound propagating in the cavity 155 to be picked up by the microphone 175. The conduit 182 opens into the cavity 155 and extends to the location of the sensor of the microphone 175.

FIG. 5 shows the two jaws 151 and 152 separated from each other. The offset structure 153 is only visible in the background, but the cavity 155 is nevertheless visible. Two annular seals 190 and 192 are present in the two halves of the annular recess 168, respectively on the surface of the jaw 151 and on the surface of the jaw 152. These seals are made of silicone and have been formed with a thin section that is to be placed in the extension of the openings 161 and 162 so as to avoid excessively compressing the plant stem.

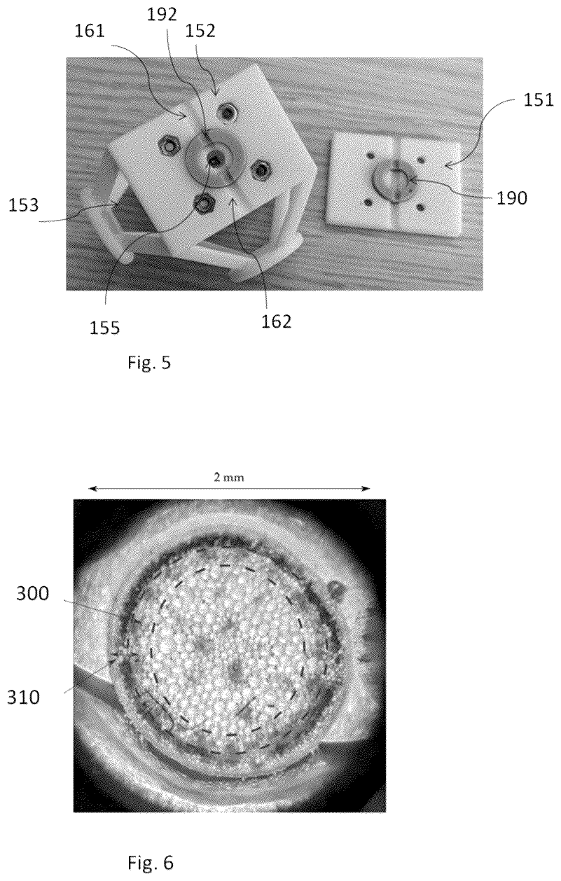

FIG. 6 is an optical microscope photograph in visible light of a cross-section of the stem of the plant used during tests. The image shows the area probed in the mid-infrared range.

The plant is a misnomer with the biological name Tradescantia zebrina. The diameter of the stem is slightly less than 2 mm. The annular area in which the phloem and xylem are located is indicated by reference 300, and the penetration depth of a laser beam at 1,036 cm⁻¹ is indicated by reference 310: it can be seen that the laser explores a significant external part of the area 300.

Liquid water is a strong absorber in the mid-infrared range, and there is a lot of water in the cells. It is therefore common practice to start by measuring water variations.

Water also directly hinders the ability to examine deep into the plant, notably beyond 150 μm behind the surface. It is therefore preferable to work on young and/or thin stems and/or roots with a diameter of less than 1 mm.

At 1,036 cm⁻¹, after water, it is likely that the signature of cell wall components, mainly made up of cellulose and lignin, will be detected.

The measurement procedure is suitable for making relative measurements and for monitoring changes in the composition being probed over time.

Indeed, it is worthwhile using the method to monitor the nature of the fluids circulating in the vascular system of the plant. As the vascular system is mainly located on the periphery of plants, it is accessible for photothermy.

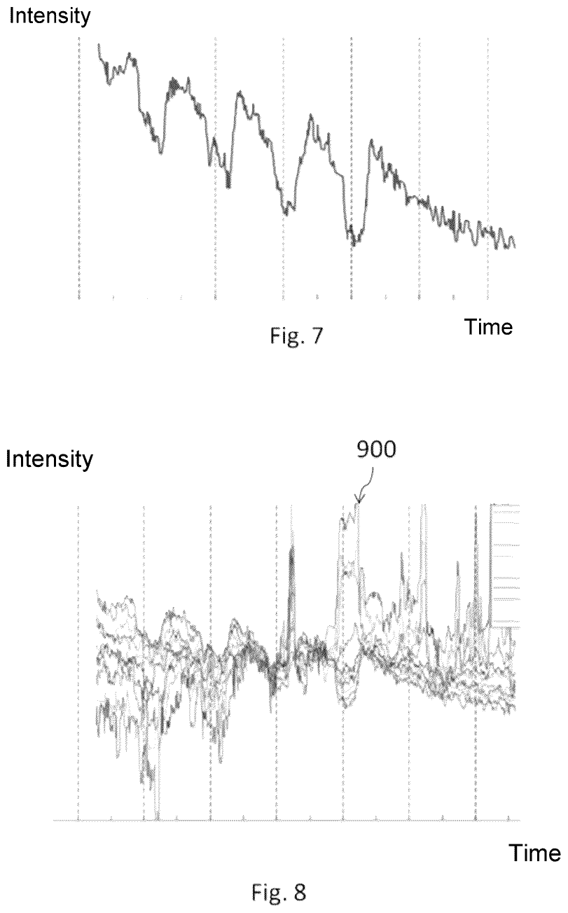

FIG. 7A series of measurements was carried out using the system described in the previous sections, shown in FIGS. 1 to 6, according to two protocols.

Protocol 1 is a continuous measurement over several days with a plant installed in the device. The presence of the stem ensures that the cavity is sealed, and the photoacoustic signal is picked up by the microphone.

Protocol 2 is a continuous measurement under the same conditions as protocol 1, but without a plant. However, the photoacoustic cell is blocked in the vicinity of the channel intended for the stem in order to ensure the generation of the photoacoustic signal. Protocol 2 acts as a reference to check that the signals detected during protocol 1 are induced by the plant and not by a drift in the system or sensitivity to changes in the environmental parameters of the experiment.

The experimental conditions for carrying out these tests are the use of an Alpes QCL laser, emitting at 1,036 cm⁻¹, with a mean laser operating point i0=0.710 A, a modulation amplitude i1=0.10 A, and a temperature regulated at 25° C. The laser power is estimated at 5 mW at the plant stem. The modulation frequencies are 107, 134, 168, 210, 162, 328, 411, 514, 643, 805, 1,007 Hz (and therefore 11 different frequencies), and the system performs a scan made up of these 11 frequencies. The scan is repeated every 4 minutes.

Each measurement of a scan involves a one-second measurement with sampling at 100 kHz. The signal is then demodulated with a slot window at the laser modulation frequency (called 1f demodulation).

The entire procedure is carried out away from natural light, but in the presence of artificial light during the day. The temperature is regulated. The sample is a stem of the tradescantia zebrina plant for protocol 1. Reading lasts 165 hours for protocol 1 and 114 hours for protocol 2.

Raw and processed data from the two experiments was examined. The data was filtered with a median filter with a width of 1h20. High-frequency noise was reduced by increasing the integration time of the synchronous detection. A median filter is a suitable solution for removing this type of noise. The data is then debiased by subtracting a common low-frequency component and slow drift, on a scale of several days, defined by averaging all the data at the various frequencies, and adding a second-order polynomial. Data that is filtered and debiased in the form of spectrograms is also of interest.

A module for monitoring the environmental conditions of the monitoring is installed. The observed temporal trends resemble xylem flow measurements taken by other means and can be linked to a plant process related to light exposure. These trends can be an increase in the speed of fluid circulation in the xylem, and thus a change in the thermal properties of the plant during the day, or an increase in the volume of water or the concentration of absorbing elements in the field of view of the laser, again during the day.

FIG. 7 shows the evolution of part of the photoacoustic signal (in this case the phase at 107 Hz in arbitrary units and after filtering and debiasing) as a function of time during a week-long experiment (the seven days of the week are marked by vertical dashed lines). Daily cycles can be observed, which are interrupted at the weekend when the plant is no longer exposed to light.

FIG. 8 shows the evolution of different parts of the photoacoustic signal (in this case the phases at the 11 aforementioned frequencies) as a function of time during an eight-day monitoring period (the days of the week are marked by vertical dashed lines).

A spatio-temporal dynamic can be observed in the phases and modules in the plant, and in particular a process that would occur from the inside to the outside (or vice versa) of the plant.

This is particularly visible in the time zone 900, which spans a little over half a day, and during which there is a signal peak that gradually shifts from high to low frequencies.

In one embodiment, the system uses a wavelength-tunable source in order to spectrally characterize the sample and to define the appropriate wavelengths according to the intended applications.

The invention has been described with the electromagnetic wave emission mode using a continuous wave with modulated intensity. However, as mentioned above, it can be implemented using a pulse method.

1. A photoacoustic method for characterizing a plant, the method comprising applying laser radiation to a material surface of the plant, recording sound waves appearing under the photothermal effect in a photoacoustic cavity positioned around said surface of the plant using a microphone, with the laser radiation having been applied to said surface through the cavity, the method wherein the plant surface is a surface of a stem, a root or a petiole of the plant, and the method comprises inserting a section of said stem, root or petiole into a support comprising the cavity equipped with a microphone sensor and a window for transmitting laser power, with sealing means being installed around the stem, root or petiole at each of the two ends of said section.

2. The photoacoustic method for characterizing a plant according to claim 1, wherein the support is made up of a set of two opposing jaws forming a vice and each provided with a seal.

3. The photoacoustic method for characterizing a plant according to claim 1, wherein the laser radiation is applied as a continuous wave with amplitude modulation at several frequencies.

4. The photoacoustic method for characterizing a plant according to claim 1, wherein the laser radiation is at a frequency in the mid-infrared range corresponding to an absorption band of a molecule of interest for monitoring the growth of the plant.

5. The photoacoustic method for characterizing a plant according to claim 1, wherein it involves continuously monitoring the plant by regularly repeating the application of the laser and the associated measurement for at least one day and one night.

6. The photoacoustic method for characterizing a plant according to claim 1, wherein the measurement is demodulated in order to identify a phase and a modulus.

7. The photoacoustic method for characterizing a plant according to claim 1, wherein the plant is alive.

8. A photoacoustic equipment for characterizing a plant, the equipment comprising means for applying laser radiation, a photoacoustic cavity configured so that the laser radiation can be applied through the cavity, and means for recording sound waves appearing under the photothermal effect in the photoacoustic cavity by means of a microphone, wherein the equipment further comprises a support comprising the cavity, with the support also comprising an insertion space for a section of a plant stem, root or petiole and sealing means for sealing the cavity at each of the two ends of said section.

9. The photoacoustic equipment for characterizing a plant according to claim 8, wherein the support is made up of a set of two opposing jaws forming a vice and each provided with a circular seal in order to form, with the opposing seal, a set of two sealed opposing passages on either side of the cavity for positioning the stem, the root or the petiole.

10. The photoacoustic equipment for characterizing a plant according to claim 8, wherein the means for applying laser radiation comprise a tunable laser or a plurality of optically coupled monochromatic lasers.
